(19) United States
(12) Patent Application Publication
(10) Pub. No.: US 2021/0205323 A1
(43) Pub. Date: Jul. 8, 2021

(54) MAINTENANCE THERAPY OF A PARP INHIBITOR IN TREATING GASTRIC CANCER

(71) Applicant: BEIGENE, LTD., Camana Bay, Grand Cayman (KY)

(72) Inventors: Beibei JIANG, Beijing (CN); Rainer K. BRACHMANN, Emeryville, CA (US); Yuting ZHANG, Emeryville, CA (US); Mitch RAPONI, Emeryville, CA (US); Heinrich FARIN, Cambridge, MA (US); Lai WANG, Beijing (CN); Hexiang WANG, Beijing (CN)

(21) Appl. No.: 17/058,812

(22) PCT Filed: May 31, 2019
(86) PCT No.: PCT/CN2019/089523

§ 371 Date: Nov. 25, 2020

(30) Foreign Application Priority Data
Jun. 1, 2018 (CN) PCT/CN2018/089564

(51) Int. Cl. A61K 31/551; A61K 33/243; A61K 31/555; A61P 35/00

(52) U.S. Cl. CPC A61K 31/551; A61P 35/00; A61K 31/555; A61K 33/243

(57) ABSTRACT

Disclosed herein is a method for maintenance therapy of a PARP inhibitor in treating a subject with gastric cancer (GC) comprising administering to the subject a therapeutic or maintenance effective amount of a PARP inhibitor, wherein the subject had previously received chemotherapy.

### CROSS-REFERENCE TO RELATED APPLICATIONS

This application claims the benefit of International Patent Application No. PCT/CN2018/089564 filed Jun. 1, 2018, the disclosures of which are hereby incorporated by reference in its entirety for all purposes.

### FIELD OF THE INVENTION

Disclosed herein is a method for maintenance therapy of a PARP inhibitor in treating a subject with gastric cancer (GC) comprising administering to the subject a therapeutic or maintenance effective amount of a PARP inhibitor, wherein the subject had previously received chemotherapy.

### BACKGROUND OF THE INVENTION

Gastric cancer is one of the most commonly diagnosed cancers, and is among the leading causes of cancer deaths worldwide. More than half of gastric cancer cases and deaths are estimated to occur in China, with approximately 680,000 cases and approximately 500,000 deaths in 2015 (Chen et al, 2016). Predicted gastric cancer deaths for the European Union in 2017 are approximately 55,000 (Malvezzi et al, 2017). For the United States (US), estimates for gastric cancer in 2017 are approximately 28,000 cases with 11,000 deaths (Siegel et al, 2017).

Gastric cancer is often in an advanced stage when it is diagnosed. Inoperable locally advanced and metastatic gastric cancer continues to be an incurable disease with 5-year survival rates below 10% despite various available treatment regimens, necessitating the exploration of additional and different therapeutic approaches. Standard first-line chemotherapy of advanced or metastatic GC includes administration of 5-Fluorouracil (i.e., 5-FU or capecitabine) plus a platinum analog (e.g., cisplatin or oxaliplatin or Carboplatin) (Smyth et al, 2016; NCCN 2017). First-line platinum-based therapy can result in favorable overall response rates around 30% to 50%. Triplet regimens may provide additional clinical benefit, as suggested in a meta-analysis for the addition of an anthracycline (Okines et al, 2009). Because of their added toxicities, however, they have not been uniformly adopted and are recommended only for medically fit patients with good performance status and access to frequent toxicity evaluations.

In the clinic, PARP inhibitors, including olaparib, rucaparib, niraparib, and talazoparib, have demonstrated sustained antitumor responses as a single agent in patients with BRCA1- or BRCA2-mutated tumors, while achieving a favorable safety profile. However, currently no drugs have been approved as a monotherapy for gastric cancer. In addition, the addition of a PARP inhibitor, i.e., olaparib, to second-line chemotherapy with paclitaxel in gastric cancer failed to improve overall survival (OS) compared with paclitaxel and placebo in the phase III clinical trial, which was contrary to the prior findings (https://www.jwatch.org/na46047/2018/02/15 second-line-olaparib-gastric-cancer, Feb. 15, 2018).

Although the first therapy including the first-line doublet or triplet chemotherapy may achieve complete response or partial response, the duration of first-line chemotherapy typically does not exceed 6 months even in those responding to treatment either due to the cumulative toxicities of chemotherapy or because of progressive disease (PD) (Cunningham et al, 2008; Van Cutsem et al, 2006; Hess et al, 2016). The time point of maximum tumor response to platinum-based therapy provides a unique opportunity to further improve on clinical benefit rather than wait for progressive disease and initiation of second-line therapy.

Therefore, for patients who have achieved maximum tumor reduction with first-line chemotherapy such as platinum chemotherapy, the concept of further treatment with a regimen of good tolerability is appealing. However, there are currently no approved drugs for maintenance treatment after first-line therapy of advanced or metastatic gastric cancer. There is a need of the maintenance therapy for those who has reached a complete response (CR) or partial response (PR) to the initial platinum-based chemotherapy, but have no further treatment options to consolidate or maintain the clinical benefit derived from first-line chemotherapy.

### SUMMARY OF THE INVENTION

The purpose disclosed herein is to provide a method for maintenance therapy of pamiparib in treating a subject with gastric cancer (GC) comprising administering to the subject a therapeutic or maintenance effective amount of a PARP inhibitor, wherein the subject had previously received chemotherapy. The method for maintenance therapy disclosed herein was found to provide additional clinical benefits by lengthening remission of a cancer or delaying growth of tumors in preclinical trials with well safety and tolerability.

In one embodiment, the subject is initially diagnosed with advanced or metastatic gastric cancer, or initially diagnosed with inoperable locally advanced or metastatic gastric cancer.

In one embodiment, the gastric cancer is adenocarcinoma of the stomach or gastroesophageal junction with inoperable locally advance or metastatic disease.

In one embodiment, the subject has previously received platinum-based chemotherapy as the first-line therapy. In another embodiment, the subject has previously received platinum-based chemotherapy in combination with fluoropyrimidine-based therapy as the first-line therapy.

In one embodiment, the platinum-based chemotherapy comprising the administration of cisplatin, or oxaliplatin, or carboplatin.

In one embodiment, the subject who had previously received chemotherapy is platinum-sensitive, i.e., in a complete response (CR) or partial response (PR) to the previous platinum-based chemotherapy before the initiation of the maintenance therapy. In a further embodiment, the subject, who had previously received platinum-based chemotherapy or platinum-based chemotherapy in combination with other chemotherapy, achieves a complete response. In yet another embodiment, the subject, who had previously received platinum-based chemotherapy or platinum-based chemotherapy in combination with other chemotherapy, achieves a partial response in which at least a 30% or 40% or 50% or 60% or 70% or 80% or 90% decrease in the tumor size has been achieved or 30% or 40% or 50% or 60% or 70% or 80% or 90% TGI has been achieved in response to the previous chemotherapy.

In one embodiment, the gastric cancer expresses high level of HRD. Preferably, the gastric cancer has one or more of the mutant genes involved in HRD, selected from BRCA1/2, BRCA-like genomic scarring, RAD51, PALB2, ATR, and ATM.

In one embodiment, the PARP inhibitor is any one of those disclosed in WO2013097225, which is incorporated herein by reference. In another embodiment, the PARP inhibitor is selected from olaparib, niraparib, rucaparib or pamiparib. Preferably, wherein the PARP inhibitor is pamiparib.

Pamiparib, is a potent and selective inhibitor of PARP1 and PARP2, and has been found to be well tolerated when compared with first- or second-line chemotherapy. It has excellent PARP trapping activity that is likely to be more important for antitumor activity than catalytic PARP inhibition. Pamiparib has also demonstrated robust antitumor activity in preclinical models. In the clinic, pamiparib has shown favorable PK properties, well safety and tolerability, and has achieved maximum pharmacodynamic target modulation in PBMCs at a dose level well below the recommended Phase 2 dose (10 versus 60 mg BID). In virtue of its excellent PARP trapping activity and well tolerance, pamiparib was found to be promising to provide clinical benefits as a maintenance therapy of a subject including a human patient who is in a complete response (CR) or partial response (PR) to the initial platinum-based chemotherapy, but have no further treatment options to consolidate or maintain the clinical benefit derived from first-line chemotherapy

In one embodiment, the PARP inhibitor including, but not limited to pamiparib, is administrated at a dose of 60 mg BID.

### BRIEF DESCRIPTION OF THE DRAWINGS

FIG. 1 shows efficacy of pamiparib and oxaliplatin+5-FU on tumor growth of BBGA087 patient derived gastric cancer model.

FIG. 2 shows the effect of pamiparib as maintenance therapy after oxaliplatin+5-FU treatment on tumor growth in BBGA087 model.

FIG. 3 shows the overall design of pamiparib as maintenance therapy in patients with advanced gastric cancer who have responded to first-line platinum-based chemotherapy

### DETAILED DESCRIPTION OF THE INVENTION

### Definitions

Unless specifically defined elsewhere in this document, all other technical and scientific terms used herein have the meaning commonly understood by one of ordinary skill in the art to which this invention belongs.

“Platinum-sensitive” is used to describe an individual or a disease or disorder who or which has responded well to the platinum-based chemotherapy to achieve complete response (CR) or partial response (PR) as defined by Response Evaluation Criteria in Solid Tumors (RECIST). In one embodiment, “Platinum-sensitive gastric cancer patient” or “platinum-sensitive patient” used herein refers to the patient who has reached complete response (CR) or partial response (PR) as defined by RECIST to the first-line platinum-based therapy.

Complete response (CR), as defined by Response Evaluation Criteria in Solid Tumors (RECIST), refers to disappearance of all target lesions. Any pathological lymph nodes (whether target or non-target) must have reduction in short axis to <10 mm.

Partial response (PR), as defined by Response Evaluation Criteria in Solid Tumors (RECIST), refers to at least a 30% decrease in the sum of diameters of target lesions, taking as reference the baseline sum diameters.

Progressive Disease (PD), as defined by Response Evaluation Criteria in Solid Tumors (RECIST), refers to at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progression.

Stable Disease (SD), as defined by Response Evaluation Criteria in Solid Tumors (RECIST), refers to neither sufficient shrinkage to qualify for PR nor sufficient increase to qualify for PD, taking as reference the smallest sum diameters while on study.

“Maintenance therapy” refers to a therapy, therapeutic regimen or course of therapy which is administered subsequent to an induction or initial therapy (i.e., an initial course of therapy administered to a subject with gastric cancer). In some embodiments, maintenance therapy is a part of the therapy for the treatment of gastric cancer. Maintenance therapy can be used to halt or reverse the progression of the gastric cancer), to maintain the improvement in health achieved by induction therapy and/or enhance, or consolidate the gains obtained by induction therapy so as lengthen remission of a cancer or delay growth of tumors.

“treatment” or “treating” or “therapy” is an approach for obtaining beneficial or desired clinical results, including, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. Therefore, a reduction of pathological consequence of urothelial carcinoma is also included by the term “treatment”. The methods disclosed herein encompass any one or more of these aspects of treatment.

By the term “effective amount”, or “therapeutically effective amount,” as used herein, is meant an amount that when administered to a mammal, preferably a human, mediates a detectable therapeutic response compared to the response detected in the absence of the complex. A therapeutic response, such as, but not limited to, increased overall survival, inhibition of and/or decreased tumor growth (including tumor size stasis), tumor size, metastasis, and the like, can be readily assessed by a plethora of art-recognized methods, including, e.g., such methods as disclosed herein. A “therapeutic effective amount”, or “effective amount,” is intended to qualify the amount of an agent required to detectably reduce to some extent one or more of the symptoms of a neoplasia disorder, including, but is not limited to: (1) reduction in the number of cancer cells; (2) reduction in tumor size; (3) inhibition (i.e., slowing to some extent, preferably stopping) of cancer cell infiltration into peripheral organs; (4) inhibition (i.e., slowing to some extent, preferably stopping) of tumor metastasis; (5) inhibition, to some extent, of tumor growth; (6) relieving or reducing to some extent one or more of the symptoms associated with the disorder, (7) relieving or reducing the side effects associated with the administration of anticancer agents; and/or (8) increasing, to some extent, the overall survival of a patient relative to that observed for the standard of care for a given tumor type or neoplastic disorder.

A “maintenance effective amount” is intended to qualify the amount of an agent required to detectably maintain the therapeutic benefit achieved during a therapeutic regimen, including, but not limited to (1) inhibiting an increase in the number of cancer cells; (2) inhibiting an increase in tumor size; (3) inhibiting cancer cell infiltration into peripheral organs; (4) inhibiting tumor metastases; (5) relieving or reducing to some extent one or more of the symptoms associated with the disorder; and/or (6) inhibiting a recurrence or onset of one or more of the symptoms associated with the disorder.

The term “subject” refers to an animal, including, but limited to, an ovine, bovine, ruminant, lagomorph, porcine, equine, canine, feline, rodent or primate, for example a human. Typically, the terms “subject” and “patient” are used interchangeably herein in reference, for example, to a mammalian subject, particularly a human subject.

### EXAMPLES

### Example A: Pre-Clinical Study

Study of pamiparib versus Placebo as Maintenance Therapy in BBGA087 Patient Derived Gastric Cancer Xenograft Model That Responded to Oxaliplatin Treatment

### Method

Female nod-scid mice were subcutaneously implanted with BBGA087 patient derived gastric tumor tissue fragments (3×3×3 mm3) in the right flank. After inoculation, tumor volume and body weight were measured twice weekly and calculated using the formula: V=0.5×(a×b2) where a and b were the long and short diameters of the tumor, respectively. When the average tumor size reached 200 mm3, animals were randomly assigned into 3 groups. Animals were treated with vehicle (0.5% methylcellulose, 0.5% MC), pamiparib, oxaliplatin plus 5-Fluorouracil (5-FU), respectively. BGB290 was administered at 6 mg/kg by oral gavage (p.o.) twice daily (BID), oxaliplatin was administered by intraperitoneal (i.p.) injection once per week (QW) for three weeks, and 5-FU was administered by intraperitoneal (i.p.) injection once daily with 5 days per week.

After 28 days, mice treated with oxaliplatin and 5-FU were reallocated into 2 groups according to the tumor volume, and were treated with either placebo (0.5% MC) or pamiparib as maintenance therapy.

Data is presented as mean tumor volume+standard error of the mean (SEM). Tumor growth inhibition (TGI) is calculated using the following formula:

$$\% growth inhibition = 100 \times ( 1 - ( \frac{( treated t ) - ( treated to )}{( placebo t ) - ( placebo to )} ) )$$

$$treated t = treated tumor volume at time t$$

$$treated t 0 = treated tumor volume at time 0$$

$$placebo t = placebo tumor volume at time t$$

$$placebo t 0 = placebo tumor volume at time 0$$

### Result

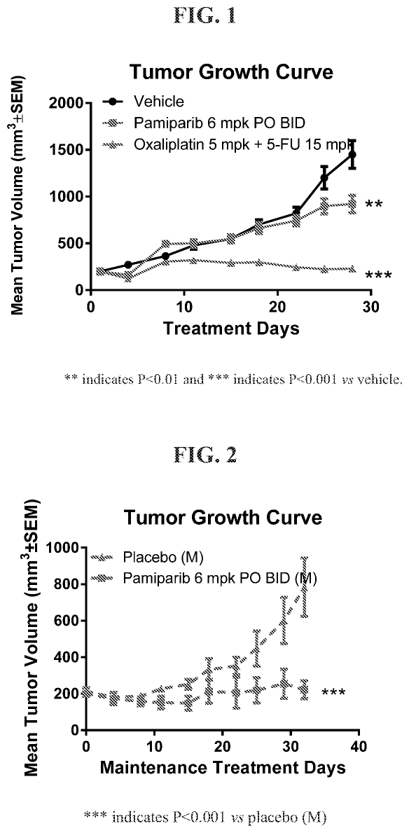

BBGA-087 gastric primary tumor model were established in female nod-scid mice using patient biopsy samples. The response of BBGA087 xenografts to pamiparib and oxaliplatin plus 5-FU was shown in FIG. 1 and Table 1. pamiparib (6 mg/kg BID) and oxaliplatin (5 mg/kg QW*3) plus 5-FU (15 mg/kg QD*5/week*3) treatment resulted in 42% and 97% statistical significant tumor growth inhibition, respectively, which suggested that BBGA087 was a responsive model to oxaliplatin.

After treatment for 28 days, oxaliplatin treated mice were further divided into two groups, the first group was treated with pamiparib (6 mg/kg BID) as maintenance therapy, the second group was treated with placebo (0.5% MC). As shown in FIG. 2 and Table 2, the tumor relapse was delayed while animals were on pamiparib maintenance therapy.

TABLE 1
Efficacy of pamiparib and oxaliplatin + 5-FU on tumor
growth of BBGA087 patient derived gastric cancer model

| | | | | Mean Tumor |
|---|---|---|---|---|
| Test | | | TGI (%) on | Volume on |
| Article | Dose(mg/Kg) | N | Day 28 | Day 28(mm3) |
| Vehicle | 0 | 10 | — | 1449 |
| pamiparib | 6 | 10 | 42 | 921 |
| Oxaliplatin + | 5 + 15 | 17 | 97 | 230 |
| 5-FU | | | | |

TABLE 2
The effect of pamiparib as maintenance therapy after oxaliplatin +
5-FU treatment on tumor grow th in BBGA087 model

| | | | | Mean Tumor |
|---|---|---|---|---|
| Test | | | TGI (%) on | Volume on |
| Article | Dosage (mg/Kg) | N | Day 32 | Day 32 (mm3) |
| Placebo (M) | 0 | 7 | — | 784 |
| pamiparib (M) | 6 | 6 | 96 | 222 |

### Example B: Clinical Study

### Methods

### Overall Design and Study Objectives

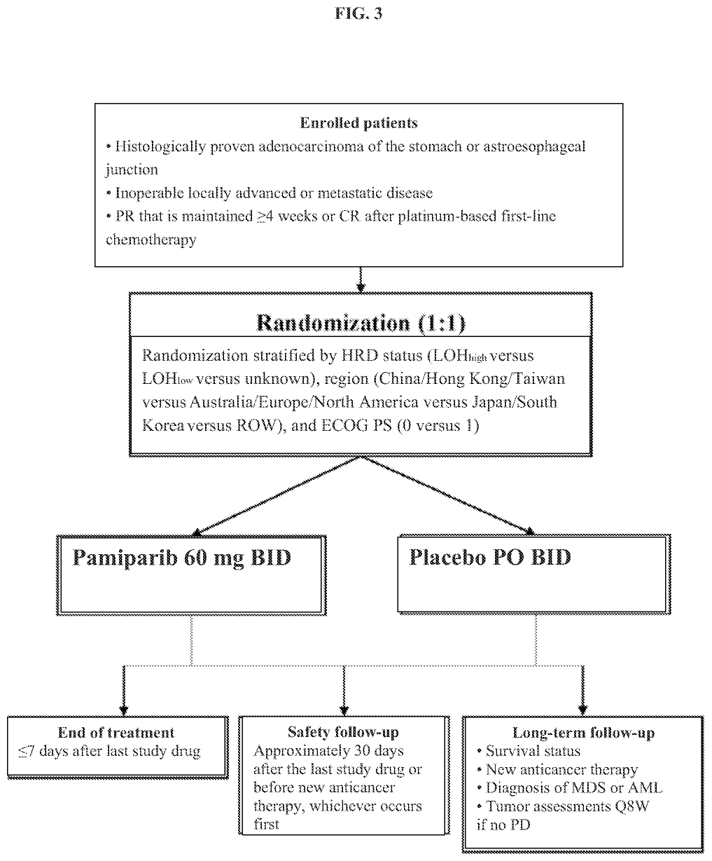

A phase 3, double-blind, placebo-controlled, randomized, multicenter study was designed to compare the efficacy, safety, and tolerability of pamiparib with placebo as maintenance therapy in patients with advanced gastric cancer who have responded to first-line platinum-based chemotherapy (FIG. 3)

The primary objective will be to evaluate the efficacy of maintenance with pamiparib versus placebo in terms of progression-free survival (PFS) assessed by a Blinded Independent Review Committee (BIRC)

Secondary objectives will include comparisons of pamiparib versus placebo for other efficacy assessments (overall survival [OS]; PFS by investigator assessment; PFS at 2 years [PFS2]; time to second subsequent treatment [TSST]; and objective response rate [ORR], duration of response [DoR], and time to response, all by investigator assessment), along with safety and tolerability
Approximately 540 patients will be enrolled at 110 study centers in Asia, Australia, Europe, and North America

### Study Population

To be eligible for participation in the study, Patients aged ≥18 years must have the following:
Histologically confirmed adenocarcinoma of the stomach or gastroesophageal junction with inoperable locally advanced or metastatic disease (patients with gastric cancer overexpressing HER2 or who received irradiation as part of prior first-line treatment will not be eligible)
Received platinum-based first-line chemotherapy with a total of ≥8 platinum-containing 14-day cycles, ≥5 platinum-containing 21-day cycles, or ≥4 platinum-containing 28-day cycles for ≤28 weeks
Achieved a partial response (PR) that is maintained for ≥4 weeks or a complete response (CR) as determined by the investigator according to Response Evaluation Criteria in Solid Tumors (RECIST) v1.1 with platinum-based first-line chemotherapy
Archival tumor tissue for central laboratory determination of HRD status
Eastern Cooperative Oncology Group performance status (ECOG PS) of 0 or 1

Patients will be excluded if they have the following:
Chemotherapy, biologic therapy, immunotherapy, investigational agent, anticancer Chinese medicine, or herbal remedies ≤14 days (or ≤5 half-lives, whichever is shorter) before randomization
Major surgical procedure, open biopsy, or significant traumatic injury ≤14 days before randomization, or are likely to need a major surgical procedure during the course of the study
Diagnosis of myelodysplastic syndrome (MDS)
Other diagnosis of malignancy
Leptomeningeal disease or brain metastasis
Cardiac chest pain or symptomatic pulmonary embolism within 28 days of randomization; or history of acute myocardial infarction, history of heart failure meeting New York Heart Association Classification III or IV, grade ≥2 ventricular arrhythmia event, or history of cerebral vascular accident, all within 6 months of randomization
Previous complete gastric resection, chronic diarrhea, active inflammatory gastrointestinal disease, or any other disease causing malabsorption syndrome
Active bleeding disorder, including gastrointestinal bleeding, as evidenced by hematemesis, significant hemoptysis, or melena within 6 months of randomization
Use within 10 days (or ≤5 half-lives, whichever is shorter) of randomization, or anticipated need for, food or drugs known to be strong or moderate cytochrome P450 (CYP) 3A inhibitors or strong CYP3A inducers

### Treatment

Patients will be randomized 1:1 (using central interactive response technology) to receive either pamiparib 60 mg twice daily or placebo, given as 28-day cycles; randomization will be stratified by genomic loss of heterozygosity status (ie, high versus low), region, and ECOG PS

Patients will receive treatment until the occurrence of progressive disease, unacceptable toxicity, death, or treatment discontinuation for other reasons *Up to two dose reductions of the study drug will be permitted during the study, and treatment can be withheld for up to approximately 28 consecutive days

Treatments and supportive care (such as antiemetic therapy, hematopoietic growth factors, and/or red blood cell/platelet transfusions) considered necessary for a patient's welfare will be permitted in keeping with the local standards of medical care

### Study Assessments and Statistical Analysis

Radiologic assessments will be centrally evaluated per RECIST v1.1 at screening and then every 8 weeks after first dose to evaluate disease progression, with tumor assessments continuing every 8 weeks in long-term follow-up for those patients without progressive disease, survival status, new anticancer therapy, and diagnosis of MDS, or acute myeloid leukemia

The primary endpoint will be PFS by BIRC assessment
Treatment groups in the intent-to-treat (ITT) population will be compared using a stratified 1-sided log-rank test at a 0.025 significance level, incorporating the randomized stratification factors; the hazard ratio (HR) and its 2-sided 95% confidence interval (CI) will be estimated
using the stratified Cox proportional hazards model
An interim analysis will be performed when 242 PFS events have occurred at approximately 23 months after start of randomization, with a final analysis performed when 363 PFS events have occurred (about 29 months post randomization)

Key secondary endpoints include an additional efficacy endpoint (such as PFS assessed by the investigator, OS, ORR, PFS2, TSST, and DoR), along with safety/tolerability
Secondary analysis of PFS will be conducted in the per-protocol analysis population and also by investigator assessment
OS will be compared across treatment groups in the ITT population using a stratified log-rank test, incorporating the randomized stratification factors, with the HR estimated using the stratified Cox proportional hazards model, median OS will be estimated using the Kaplan-Meier method
Other secondary time-to-event endpoints, such as PFS2, TSST and DoR, will be analyzed in a similar manner to OS
ORR will be reported for the ITT population, with treatment groups compared using a Cochran-Mantel-Haenszel score test

Safety will be monitored throughout the study (Day 1 of each cycle, on Day 15 of Cycles 1 and 2, and as needed), with safety assessments including adverse event monitoring, physical examinations, vital sign measurements, electrocardiograms, and clinical laboratory tests

Adverse events will be documented during treatment for approximately 30 days after the last dose of study drug or until initiation of new anticancer therapy

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

1. A method for providing maintenance therapy in a subject with gastric cancer comprising administering to the subject a therapeutic or maintenance effective amount of a PARP inhibitor, wherein the subject had previously received chemotherapy.

2. The method of claim 1, wherein the subject is initially diagnosed with advanced or metastatic gastric cancer.

3. The method of claim 2, wherein the subject is initially diagnosed with inoperable locally advanced or metastatic gastric cancer.

4. The method of claim 1, wherein the gastric cancer is adenocarcinoma of the stomach or gastroesophageal junction with inoperable locally advance or metastatic disease.

5. The method of claim 1, wherein the subject has previously received platinum-based chemotherapy.

6. The method of the claim 5, wherein the subject has previously received first line platinum-based chemotherapy.

7. The method of any one of claim 6, wherein the subject has previously received platinum-based chemotherapy in combination with fluoropyrimidine-based therapy.

8. The method of claim 7, wherein the subject received cisplatin, oxaliplatin, or carboplatin as the platinum-based chemotherapy.

9. The method of claim 8, wherein the subject had a complete response (CR) or a partial response (PR) to the previous platinum-based chemotherapy before the initiation of the maintenance therapy.

10. The method of claim 9, wherein the gastric cancer expresses high level of homologous recombination deficiency (HRD).

11. The method of claim 10, wherein the gastric cancer has one or more of the mutant genes involved in HRD, selected from the group consisting of BRCA1/2, BRCA-like genomic scarring, RAD51, PALB2, ATR, and ATM.

12. The method of claim 1, wherein the PARP inhibitor is olaparib, niraparib, rucaparib or pamiparib.

13. The method of claim 1, wherein the PARP inhibitor is pamiparib.

14. The method of claim 13, wherein pamiparib is administered at a dose of 60 mg BID.
